(19) United States
(12) Patent Application Publication
(10) Pub. No.: US 2023/0210211 A1
(43) Pub. Date: Jul. 6, 2023

(54) A CONDUCTIVE APPARATUS FOR FOOTWEAR

(71) Applicant: EARTHLING 3.0 PTY LTD, South Australia (AU)

(72) Inventor: George IOANNOU, South Australia (AU)

(21) Appl. No.: 17/800,615

(22) PCT Filed: Feb. 22, 2021
(86) PCT No.: PCT/AU2021/050150

§ 371 Date: Aug. 18, 2022

(30) Foreign Application Priority Data
Feb. 21, 2020 (AU) 2020900495

(51) Int. Cl. A43B 7/36; H01R 4/04

(52) U.S. Cl. CPC A43B 7/36; H01R 4/04; A61N 1/14

(57) ABSTRACT

A conductive apparatus, including a conductive first layer, a second adhesive layer and a third protective layer. Each of the layers includes a web joining two tabs at either, the first tab of the conductive first layer adapted to be attached to the inside of a shoe and the second tab adapted to be attached to the sole of the shoe thus exposing the protective layer. The protective layer includes at least one aperture in each tab to accommodate a micro metal component.

### TECHNICAL FIELD

The present disclosure generally relates to conductive apparatuses, in particular wearable conductive apparatuses for footwear.

### BACKGROUND OF THE INVENTION

The term “earthing” or “grounding” refers to a human body being in contact with the surface of the earth by barefoot exposure outdoors or using special indoor systems connected to the earth. Such contact enables transfer of the earth's free electrons into and/or from the electrically conductive human body.

Studies indicate that grounding may provide beneficial effects to the body, e.g., improving blood flow, reducing inflammatory response, reducing muscle soreness, and/or decreasing post-exercise muscle damage.

Some devices have been proposed to facilitate grounding human bodies to the earth. For example, one device allows a user to be grounded by putting the user's feet on a conductive mat, the mat being connected into the ground port of an electric power outlet via conductive wires; however, when using this device, in order to maintain in contact with the mat, the user is required to stand or sit in a fixed location, which may be inconvenient or undesirable.

Whilst earthing straps have been found to be effective, they lack durability and in some instances the ability to be printed on for branding purposes. They may also be fragile in that use even over a short period of time may render the straps ineffective.

It is desired to address or ameliorate one or more disadvantages or limitations associated with the prior art, or to at least provide a useful alternative.

### SUMMARY OF THE INVENTION

In accordance with the present invention there is proposed a conductive apparatus, including:

a conductive first layer;
a second adhesive layer;
a third protective layer;
wherein each of the layers includes a web joining two tabs at either end thereof;
the first tab of the conductive first layer adapted to be attached to the inside of a shoe and the second tab adapted to be attached to the sole of the shoe thus exposing the protective layer;
wherein the protective layer includes at least one aperture in each tab to accommodate a micro metal component.

In preference there is two or more apertures in the protective layer.

### BRIEF DESCRIPTION OF THE DRAWINGS

Some embodiments of the present invention are hereinafter further described, by way of example only, with reference to the accompanying drawings, in which:

FIG. 1 is a top view of a conductive apparatus according to one embodiment;

FIG. 2 is a top view of the conductive apparatus according to a second embodiment;

FIG. 3 is a perspective view of the conductive apparatus according to the first embodiment;

FIG. 4 is an exploded view of the conductive apparatus;

FIG. 5 is an enlarged view of one end of the conductive apparatus; and

FIG. 6 is a bottom view of the conductive apparatus attached to the underside of a shoe.

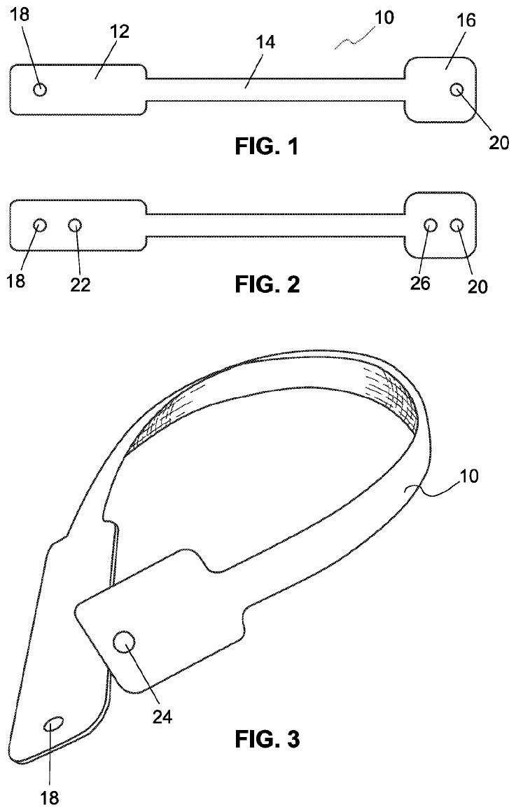

### DETAILED DESCRIPTION

Described herein is a conductive apparatus in the form of a conductive device 10. As shown in the figures, the conductive device 10 includes a first tab 12 formed at one end of the conductive device 10, a web 14 connecting the first conductive portion 12 to a second tab 16 at the end of the conductive device 10, making the whole device 10 conductive.

The conductive device 10 is used with a footwear garment 36. The conductive device 10 is manually attachable to the footwear garment, i.e., can be attached by hand. The conductive device 10 is removably attachable to the footwear garment 36. The footwear garment may be any suitable type of footwear.

As is well known in the art including the applicants own Australian patent application 2019284138 whose description is incorporated by reference herein, the first tab 12 attaches to an inner portion of the footwear garment 36, while the second tab 16 attaches to an outer portion on an underside of the footwear garment 36.

The tabs attach to the footwear garment 36 adhesively.

The first conductive tab 12 and the second tab 16 are electrically connected by, with, and through the web 14, which at least partly extends along an outside of the footwear garment 36.

As shown in the figures, the web 14 has an elongate shape, i.e., is elongated. The first tab 12 and the second tab 16 each have a greater width than the web 14. The first tab 12 has a greater length than the second tab 16, which may facilitate providing a reliable electrical connection between the first tab 12 and the human body. In this disclosure, the term “length” refers to the dimension in the device longitudinal direction, and the term “width” refers to the dimension in the transverse direction, the third orthogonal direction is “thickness”.

As shown in the figures, both the first tab 12 and the second tab 16 are substantially flat or “planar”, which may increase the user's comfort while wearing the conductive device 10, and may reduce or prevent any obstruction to the user's movement caused by the conductive device 10.

The conductive device 10 is in the form of a flexible strap that is adaptable to at least part of a contour the footwear garment 36.

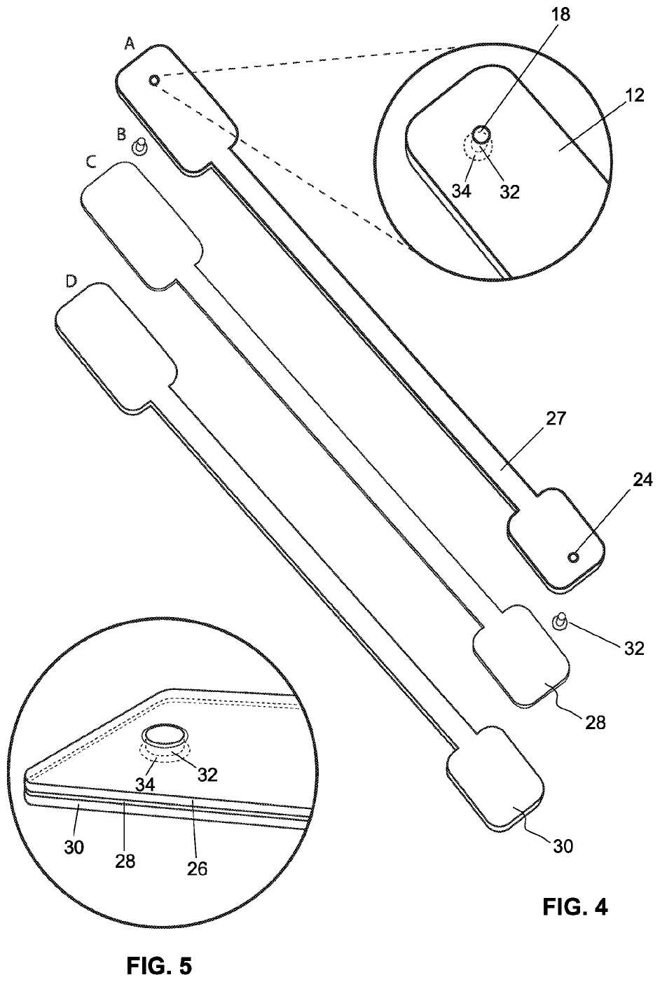

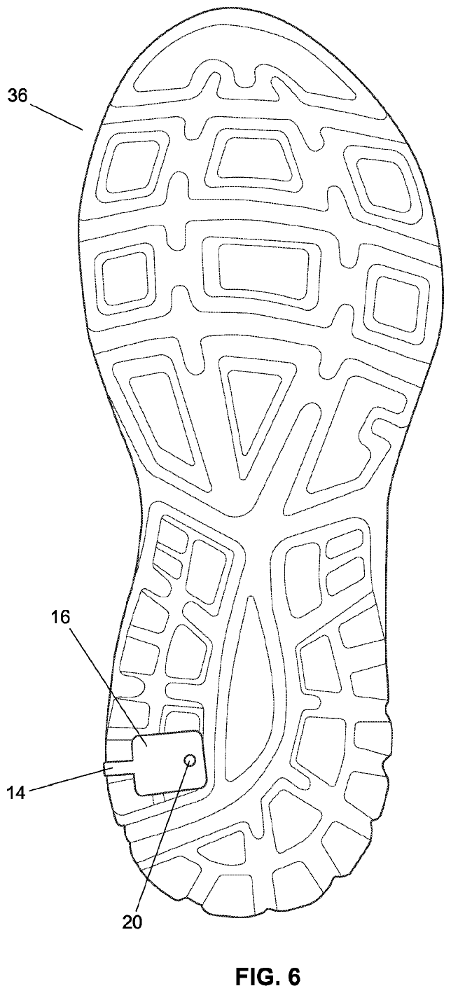

As shown in FIG. 4, the strap includes plurality of layers, i.e.:
a. a protective layer 27;
b. an adhesive layer 28; and
c. a conductive layer 30.

For details of various constructions of the layers the reader is referred to the abovementioned AU patent application.

In the present embodiment, the conductive device 10 is adhesively attachable to the footwear garment 36. Alternatively, in some embodiments, at least one of the first tab 12 and the second tab 16 may be attachable to the footwear garment 36 by other means. For example, the footwear garment may have loops, e.g., a fluffy inner and/or outer surface (e.g., the footwear may be a sock): accordingly, instead of an adhesive surface, one surface of the first tab 12 and/or the second tab 16 may have hooks that engage with the loops (e.g., like Velcro or hook-and-loop tape), so as to attach to the footwear garment through a hook-and-loop engagement.

Further, in the present embodiment, the whole conductive device 10 is adhesively attachable to the footwear garment 36. Alternatively, in some embodiments, it may be that only the first and second tabs are adhesively attachable to the footwear.

The protective layer apparatus includes apertures 18 and 24. The apertures extend through the layer 27 and are designed so that a micro metal component extends through the layer 27. The component is a micro metal component that is of a cylindrical configuration with the top of the component including effectively a pin 32 and the bottom a base 34. The pin is typically of 1.5 mm in diameter whilst the bottom is typically of 3 mm diameter. It is not intended that the metal component be of a cylindrical configuration. The purpose of the bottom being of a greater diameter than the top is to ensure that the metal component does not protrude through the layer 27.

The vertical extend of the two components 32 and 34 may vary.

In a further embodiment as illustrated in FIG. 2 the apparatus may include more than one aperture on the protective layer. This is effectively ensuring if by whatever means one of the metal components fails or is blocked there is another one that can perform the same function.

Finally the strap is designed so that the protective layer, that is exposed, can be used to print material on, such as advertising.

### DRAWING COMPONENTS

The drawings include the following integers:
10 conductive device
12 first tab
14 web
16 second tab
18 aperture in first tab of protective layer
20 aperture in second tab or protective layer
22 second aperture in first tab
26 second aperture in second tab
27 protective layer
28 adhesive layer
30 conductive cover
32 top of metal component
34 bottom of metal component
36 footwear

Thus in reference to the present application we state the following. The present disclosure generally relates to a conductive apparatus, in particular used with footwear.

The first manufacturing step is to establish the design of the conductive footwear apparatus that one desires the protective cover to be applied to. Once this is established, the next step is to cut specially designed micro holes into the protective layer in appropriate positions. There are only two holes required per protective layer, but more can be made, and the positions of these holes are to be lined up with the conductive layer where it is to come into contact with the ground and a second hole positioned where the conductive apparatus is to come into contact with the body of the person wearing the apparatus.

The holes are typically cut in two phases, each phase accounting account for half the thickness of the sheet. (so for a 0.2 mm thick sheet, one hole will be cut at 0.1 mm deep, and another hole will be cut at 0.1 mm deep to make one 0.2 mm deep hole). Firstly a smaller hole, roughly 1.5 mm in diameter will be cut at the top and then a larger hole, roughly 3 mm in diameter will be cut at the bottom. These holes are generally cut using a laser or specifically designed cutting tool.

The next step will be to cut the micro metal component to place into the holes. The first step is to use a sheet of metal, (stainless steel etc) of the same thickness as the holes they will fill (0.2 mm). The sheet is then cut in two stages. Firstly, a CNC router or laser will face cut a 3 mm diameter, 0.1 mm deep circle doughnut on the face of the sheet of metal, leaving a centre portion of the doughnut raised without cutting into it. This centre portion is to be a diameter of 1.5 mm and will remain 0.2 mm thick as the original sheet of metal. The second step is to cut the outside perimeter of the 3 mm diameter doughnut all the way through the 0.2 mm thick sheet of metal in order to remove the component from the metal sheet. This is done using a disc cutting tool or CNC router or laser. Once it is cut the component presents as a 3 mm diameter, 0.1 mm thick metal disc with a raised 1.5 mm diameter 0.1 mm raised portion in the centre. The overall thickness of the component is 0.2 mm.

Once complete, the micro metal components can be dipped in an electrically conductive glue and placed in the identically shaped holes in the protective cover. Once the micro metal components are applied, the underside of the sheet can be laminated with a conductive adhesive.

In a preferred embodiment the next step is to prime the top of the protective cover to accept any type of print. Once the sheet is primed and/or printed it can then be cut to the desired shape and laminated to the conductive apparatus for footwear. It can also be laminated in sheet form during the manufacturing process and cut together with the conductive apparatus.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Many modifications will be apparent to those skilled in the art without departing from the scope of the present invention.

1. In accordance with the present invention there is proposed a conductive apparatus, including:
a conductive first layer;
a second adhesive layer;
a third protective layer;
wherein each of the layers includes a web joining two tabs at either end thereof;
the first tab of the conductive first layer adapted to be attached to the inside of a shoe and the second tab adapted to be attached to the sole of the shoe thus exposing the protective layer;
wherein the protective layer includes at least one aperture in each tab to accommodate a micro metal component.

2. In preference there are two or more apertures in the protective layer.
